(19) United States
(12) Patent Application Publication
(10) Pub. No.: US 2007/0128280 A1
(43) Pub. Date: Jun. 7, 2007

(54) Oral osmotic drug delivery system

(76) Inventor: Hasmukh B. Patel, Edison, NJ (US)

Correspondence Address: ARENT FOX PLLC, 1050 CONNECTICUT AVENUE, N.W., SUITE 400, WASHINGTON, DC, 20036, US

(21) Appl. No.: 11/292,305

(22) Filed: Dec. 2, 2005

(51) Int. Cl. A61K 31/455; A61K 9/24

(52) U.S. Cl. 424/473; 514/355

(57) ABSTRACT

An oral osmotic delivery system and methods of using the oral osmotic delivery system that provide controlled and sustained delivery of a drug. The osmotic drug delivery system includes a drug layer comprising a drug and a first hydrophilic polymer wherein the drug layer comprises about 14.8% to about 100% fines; an osmotic layer comprising a second hydrophilic polymer; and an outer coating wherein the outer coating substantially surrounds the drug layer and the osmotic layer and wherein the outer coating comprises at least one opening. The oral osmotic delivery system provides a 16-hour cumulative dissolution (Q) value of 85% or more and % release rates (RR) that are therapeutically effective.

### FIELD OF THE INVENTION

The present invention relates to an oral osmotic delivery system comprising a drug composition and, more particularly, an oral osmotic delivery system and methods of using the oral osmotic delivery system that provide controlled and sustained delivery of a drug.

### BACKGROUND OF THE INVENTION

Pharmaceutical compositions intended for oral administration are typically solid dosage forms (e.g., tablets) or liquid preparations (e.g., solutions, suspensions, or elixirs). The usefulness of an oral formulation, however, requires that the active agent be bioavailable at therapeutic levels over a specific time period of time. For example, for ailments requiring multiple doses of a particular drug, the blood levels of the drug need to be maintained above its minimum effective level and below its minimum toxic level to obtain the desired therapeutic effects, to avoid undesired toxic effects, and to minimize side effects. Thus, methods of controlled drug delivery are desirable to achieve substantially constant blood levels of the active ingredient, as compared to the uncontrolled fluctuations observed when multiple doses of immediate release conventional dosage forms are administered to a patient. Further, methods of controlled drug delivery may not only reduce the frequency of dosing, but may also reduce the severity and frequency of side effects.

Efforts have been made to develop new pharmaceutically viable and therapeutically effective controlled drug delivery systems. In particular, orally administered controlled drug delivery systems because of the ease of administration via the oral route as well as the ease and economy of manufacture of oral dosage forms such as tablets and capsules. Various oral controlled drug delivery systems based on different release mechanisms have been developed, for example, dissolution controlled systems, diffusion controlled systems, ion-exchange resins, osmotically controlled systems, erodible matrix systems, pH-independent formulations and swelling controlled systems.

Osmotic dosage forms utilize osmotic pressure to absorb fluid into through a semi-permeable wall, which permits free diffusion of fluid but not the drug or the osmotic agent, and pushes the drug out of a hole or opening in the dosage form. A significant advantage to osmotic systems is that operation is pH-independent. A review of osmotic delivery forms is provided in Santus et al., Journal of Controlled Release 35, 1-21 (1995), the disclosure of which is hereby incorporated by reference in its entirety. In addition, U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850 are each related to directed to osmotic dosage forms, the disclosures of which are hereby incorporated by reference in their entirety.

U.S. Pat. Nos. 4,816,263; 4,950,486; 4,946,687; and 5,030,456 relate to a method for treating a cardiovascular disease by providing an oral dosage form that can deliver isradipine over a prolonged period of time at a controlled rate and in a constant dose per unit time.

Sastry et al. disclose that various factors, such as, orifice size, coating thickness, amount and nature of polymeric excipients, and amount of osmotic agent influence the drug release from Gastrointestinal Therapeutic Systems (GITS). In particular, that the in-vitro atenolol release rate of atenolol GITS, coated with CA pseudolatex is influenced by orifice size, % coating weight gain and amount of Carbopol 934P. Furthermore, that drug release under constrained conditions is influenced by the following factors, with decreasing order of importance: % coating weight gain>Carbopol 934P>Polyox N80>Carbopol 974P>Polyox 303>amount of sodium chloride>orifice size. Sastry et al., Pharm Acta Helv. 73(2):105-12 (1998).

Although osmotic dosage forms have been disclosed, there remains a need in the art for an oral osmotic delivery system with improved performance. In particular, an oral osmotic delivery system that can provide reliable and effective controlled release of a drug after administration to a subject.

### SUMMARY OF THE INVENTION

The present invention relates to an oral osmotic delivery system comprising a drug composition and, more particularly, an oral osmotic delivery system and methods of using the oral osmotic delivery system that provide controlled and sustained delivery of a drug.

In accordance with the present invention there is provided an osmotic drug delivery system for delivering a drug to a subject comprising: a drug layer comprising a drug and a first hydrophilic polymer wherein the first hydrophilic polymer has a molecular weight of about 40,000 to about 750,000 and wherein the drug layer comprises about 14.8% to about 100% fines; an osmotic layer comprising a second hydrophilic polymer wherein the second hydrophilic polymer has a molecular weight of about 1,000,000 to about 15,000,000; and an outer coating wherein the outer coating substantially surrounds the drug layer and the osmotic layer and wherein the outer coating comprises at least one opening which allows the drug to be released from the drug layer.

In an exemplary embodiment, the drug layer comprises about 20% to about 50% fines. In further exemplary embodiments, the drug layer comprises over 28% to about 40% fines.

In another exemplary embodiments, the drug in the drug layer comprises a dihydropyridine. In a preferred embodiment, the drug in the drug layer is isradipine.

In yet another exemplary embodiment, the drug delivery system releases at least 85% of the drug in 16 hours under the following dissolution conditions: USP dissolution test <711>, Apparatus 2, 50 rpm, 500 to 1000 ml of 0.2% N,N-dimethyl dodecylamine N-oxide (LDAO) or equivalent solvent, temperature 37° C.±0.5° C. In a further embodiment, at least 90% of the drug is released.

Other novel features and advantages of the present invention will become apparent to those skilled in the art upon examination of the following or upon learning by practice of the invention.

### BRIEF DESCRIPTION OF THE DRAWINGS

FIG. 1 shows an exemplary bi-layer tablet according to the present invention;

FIG. 2 shows the particle size distributions (PSD) of the exemplary osmotic granulations of the present invention;

FIG. 3 shows the particle size distributions (PSD) of the exemplary osmotic granulations of the present invention;

FIG. 4 is a graph that shows the effect of % coarse particles of the drug layer on 16-hour cumulative drug dissolution;

FIG. 5 is a graph that shows the effect of % coarse particles of the drug layer on % release rate;

FIG. 6 is a graph that shows the effect of % fines of the drug layer on 16-hour cumulative drug dissolution;

FIG. 7 is a graph that shows the effect of % fines of the drug layer on % release rate;

FIG. 8 is a graph that shows the relationship between fines and coarse particles of the drug layer;

FIG. 9 shows the particle size distributions (PSD) of the exemplary osmotic granulations of the present invention; and

FIG. 10 shows the particle size distributions (PSD) of the exemplary osmotic granulations of the present invention.

### DETAILED DESCRIPTION

The present invention relates to an oral osmotic delivery system comprising a drug composition and, more particularly, an oral osmotic delivery system and methods of using the oral osmotic delivery system that provide controlled and sustained delivery of a drug to a subject.

The oral osmotic delivery system of the present invention comprises a drug layer comprising a drug and a hydrophilic polymer wherein the hydrophilic polymer has a molecular weight of about 40,000 to about 750,000 and wherein the drug layer comprises about 14.8% to about 100% fines; an osmotic layer comprising a hydrophilic polymer wherein the hydrophilic polymer has a molecular weight of about 1,000,000 to about 15,000,000; and an outer coating wherein the outer coating substantially surrounds the drug layer and the osmotic layer and wherein the outer coating comprises at least one opening which allows the drug to be released from the drug layer.

According to one exemplary embodiment of the present invention, the oral osmotic delivery system may be a bi-layer tablet that comprises a drug layer and an osmotic layer, as shown in FIG. 1. The bi-layer tablet may further comprise an outer coating and an opening in the outer coating, which allows controlled release of the drug from the tablet. For example, as described below, the outer coating may be semi-permeable such that the hydrated drug is pushed out of the opening in the tablet as a result of the pressure created by swelling of the osmotic layer in presence of fluid which enters into the system through the semi-permeable outer coating.

It has been surprisingly discovered that the particle size of the drug layer, defined as fines (i.e., particles passing through a US #120 mesh sieve; or particles of equal to or less than 125 microns in size), has a significant and unexpected impact on the 16-hour cumulative dissolution (Q) and the % Release Rate (RR). In particular, it has been surprisingly discovered that the Q value is directly proportional to the amount of fines in the drug layer. In addition, it has also been surprisingly discovered that the RR value is directly proportional to the amount of fines in the drug layer. This discovery applies not only to the total percentage of fines in the drug layer, but also the percentage of fines of hydrophilic polymer in the drug layer.

Thus, the oral osmotic delivery system of the present invention provides sustained, and preferably uniform, release for a prolonged period of time within a sustained release time period. For example, the oral osmotic delivery system of the present invention provides a 16-hour cumulative dissolution (Q) value of 85% or more and more preferably 90% or more, under the following dissolution conditions: USP dissolution test <711>, Apparatus 2, 50 rpm, 500 to 1000 ml of 0.2% N,N-dimethyl dodecylamine N-oxide (LDAO) or equivalent solvent, temperature 37° C.±0.5° C. In addition, the oral osmotic delivery system of the present invention further provides % release rates (RR) that are therapeutically effective. The drug “release rate” refers to the percent of the labelled amount of drug released from a dosage form per unit time, e.g., % of drug released per hour (%/hr).

The delivery system of the present invention further provides sustained, preferably uniform, release of a drug for a continuous period of time of at least 6-12 hours or more and, more preferably, 16 hours or more. For example, the exemplary osmotic delivery system typically begins releasing the drug at a uniform release rate within about 2 to about 6 hours following administration and the release of the drug continues for at least 16 hours or more, until at least about 85% and more preferably at least about 90% of the drug is released from the dosage form. In exemplary embodiments, the delivery system of the present invention provides an average hourly release rate that varies by no more than about 30% and preferably no more than about 25% from either the preceding or the subsequent average hourly release rate, for at least 6 continuous hours during the period in which drug is released.

Thus, the oral osmotic delivery system of the present invention provides sustained release of a drug over a continuous period of time after administration to a subject. Furthermore, the delivery systems of the present invention may provide blood plasma drug concentrations in the subject that are less variable over a prolonged period of time than those obtained with other dosage forms known in the art. In addition, when the dosage forms of the present invention are administered on a continuous once-a-day basis, therapeutically effective concentrations of the drug is provided.

According to the present invention, the oral osmotic delivery system includes a drug layer that comprises a drug and hydrophilic polymer. Any drug known in the art, typically provided through oral administration, may be used according to the present invention. For example, in some embodiments according to the present invention, the drug in the drug layer comprises a dihydropyridine. In a preferred embodiment the drug in the drug layer comprises isradipine.

The oral osmotic delivery system according to the present invention further includes a hydrophilic polymer with a molecular weight of about 40,000 to about 750,000. Hydrophilic polymers that may be used in the drug layer of the present invention include any polymers that interact with water and aqueous biological fluids and swell or expand to a high degree, typically exhibiting a 2-50 fold volume increase. In some embodiments according to the present invention, the hydrophilic polymer of the drug layer comprises a poly(alkylene oxide), including, but not limited to, poly(ethylene oxide), poly(methylene oxide), poly(butylene oxide) and poly(hexylene oxide), preferably at 100,000 to 750,000 molecular weight. In other embodiments according to the present invention, the hydrophilic polymer of the drug layer comprises a poly(carboxymethylcellulose), including, but not limited to, poly(alkali carboxymethylcellulose) such as poly(sodium carboxymethylcellulose), poly(potassium carboxymethylcellulose) and poly(lithium carboxymethylcellulose), preferably at 40,000 to 400,000 molecular weight. In some embodiments according to the present invention the hydrophilic polymer comprises (poly)hydroxypropylmethyl cellulose, (poly)hydroxypropyl cellulose, or (poly)vinyl alcohol. In some embodiments according to the present invention the hydrophilic polymer comprises poly(ethylene oxide) with a molecular weight of from about 100,000 to about 300,000. In a preferred embodiment, the poly(ethylene oxide) has a molecular weight of about 200,000.

In one embodiment according to the present invention, the drug layer of the oral osmotic delivery system comprises about 2 weight percent to about 30 weight percent of isradipine and from about 30 weight percent to about 95 weight percent of a polyethylene oxide.

Any method known in the art may be used to produce the particles of the drug layer of the present invention including, e.g., granulation, spray drying, sieving, lyophilization, crushing, grinding, jet milling, micronizing and chopping. The size of the particle can be ascertained by screening, including a grizzly screen, a flat screen, a vibrating screen, a revolving screen, a shaking screen, an oscillating screen and a reciprocating screen. The processes and equipment for preparing drug and carrier particles are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990), the disclosures of which are hereby incorporated by reference in their entirety.

According to the present invention, the oral osmotic delivery system includes an osmotic layer comprising a hydrophilic polymer having a molecular weight of about 1,000,000 to about 15,000,000. Hydrophilic polymers that may be used in the osmotic layer of the present invention include any polymers that interact with water and aqueous biological fluids and swell or expand to a high degree, typically exhibiting a 2-50 fold volume increase. For example, hydrophilic polymers that may be used in the osmotic layer of the present invention may include the same types of polymers indicated above for the drug layer. Preferably, the hydrophilic polymer is selected from a poly(alkylene oxide), including, but not limited to, poly(ethylene oxide), and poly(alkali carboxymethylcellulose). In preferred embodiments, the hydrophilic polymer in the osmotic layer has a molecular weight of about 7,000,000.

In some embodiments the hydrophilic polymers of the present invention comprise polymers that form hydrogels, such as, but not limited to, an acidic carboxypolymer, e.g., carboxypolymethylene, or a carboxyvinyl polymer, a polyacrylamide, a polyacrylic acid, or an acrylate polymer polysaccharide. Representative polymers that form hydrogels are known in the art and have been disclosed, for example, in U.S. Pat. Nos. 3,865,108; 4,002,173; 4,207,893; and in Scott and Roff, Handbook of Common Polymers, Chemical Rubber Co., Cleveland, Ohio, the disclosures of which are hereby incorporated by reference in their entirety.

According to the present invention, the oral osmotic delivery system comprises an outer coating that substantially surrounds the drug layer and the osmotic layer. In preferred embodiments, the out coating is semi-permeable such that it allows the passage of external fluids such as water and biological fluids, and is substantially impermeable to the passage of the drug included in the drug layer. One skilled in the art will appreciate that the outer coating may be comprised of various materials and be within the scope of the present invention. For example, the outer coating may comprise polymers, such as, semipermeable homopolymers or semipermeable copolymers. In some embodiments according to the present invention, the outer coating may comprise a cellulosic polymer. For example, the cellulosic polymer may comprise cellulose acylate, cellulose diacylate, cellulose triacylate, cellulose acetate, cellulose diacetate and cellulose triacetate.

In a further embodiment, the outer coating may also comprise a flux-regulating agent to assist in regulating the fluid permeability through the outer coating. The flux-regulating agent can be a flux-enhancing agent or a flux-decreasing agent. Agents that produce a marked increase in permeability to fluid such as water are often essentially hydrophilic, while those that produce a marked decrease to fluids such as water are essentially hydrophobic. The flux regulators that may be used according to the present invention may include, e.g., polyhydric alcohols, polyalkylene glycols, polyalkylenediols and polyesters of alkylene glycols.

According to the present invention, the outer coating that substantially surrounds the drug layer and the osmotic layer comprises at least one opening. The opening allows the uniform release of the drug from the delivery system and may include, for example, a passageway, an aperture, an orifice, or a bore. The opening in the outer coating may be formed by drilling, including mechanical and laser drilling. In some embodiments, the opening may be formed from a substance or polymer that erodes, dissolves or is leached from the outer coating to leave an opening that allows the uniform release of the drug from the delivery system.

The present invention also provides methods of using an oral osmotic delivery system to provide controlled and sustained delivery of a drug. For example, the present invention provides a method of delivering a drug to a subject comprising administering to the subject an osmotic drug delivery system as described above.

After oral ingestion of the oral osmotic delivery system of the present invention, the osmotic activity gradient across the semi-permeable outer coating causes gastric fluid to be absorbed through the outer coating. The drug within the drug layer is then released through the opening in the outer coating. As the drug is released gastric fluid is continually absorbed which, in turn, causes release of more of the drug. Thus, the drug is released in a sustained and continuous manner over an extended time period.

The following examples are merely illustrative of the present invention and should not be construed as limiting the scope of the invention in any way as many variations and equivalents that are encompassed by the present invention will become apparent to those skilled in the art upon reading the present disclosure.

### EXAMPLES

As described below, the following examples show that the particle size of the drug layer, defined as fines, has a significant and unexpected impact on the 16-hour cumulative dissolution (Q) and the % Release Rate (RR). In particular, the examples show that the Q value is directly proportional to the amount of fines in the drug layer. In addition, the examples show that the RR value is also directly proportional to the amount of fines in the drug layer. In each test, the following dissolution method was employed: USP dissolution test <711>, Apparatus 2, 50 rpm, 500 to 1000 ml of 0.2% N,N-dimethyl dodecylamine N-oxide (LDAO) or equivalent solvent, temperature 37° C.±0.5° C.

Table 1 shows the dissolution profiles for tablet batches as a function of % fines. The % dissolution at 4, 8 and 12-hour time points were determined and the Q value and RR value were calculated. Table 2 shows the statistical analysis of the relationship between Q and fines. This statistical analysis demonstrates that the relationship between Q and % fines is statistically significant (t=4.9, F=24.2 and P<0.01).

TABLE 1
Dissolution Profiles of the Tablet

| | Drug | | | | | |
|---|---|---|---|---|---|---|
| Tablet | Layer | Time (h) | | | Dissolution | |
| Batch# | % Fine | 4 | 8 | 12 | % Q | % RR |
| 3041648AR | 16.0 | 17.2 | 53.0 | 83.3 | 89.7 | 8.26 |
| 3041648BR | 16.0 | 18.6 | 56.5 | 87.0 | 96.0 | 8.55 |
| 3041648CR | 16.0 | 16.9 | 54.7 | 85.4 | 91.4 | 8.56 |
| 3041648RD | 16.0 | 14.8 | 49.6 | 75.5 | 84.7 | 7.78 |
| 3041648ER | 16.0 | 16.8 | 53.3 | 83.1 | 88.9 | 8.29 |
| 3042050AR | 21.0 | 19.9 | 59.7 | 90.6 | 97.2 | 8.84 |
| 3042050BR | 21.0 | 18.5 | 54.0 | 84.6 | 92.6 | 8.27 |
| 3042050DR | 21.0 | 22.1 | 63.7 | 94.6 | 99.2 | 9.06 |
| 3042051RA | 19.0 | 15.9 | 49.1 | 79.7 | 88.9 | 7.97 |
| 3042051BR | 19.0 | 20.8 | 59.4 | 88.9 | 95.0 | 8.51 |
| 3042051RC | 19.0 | 16.6 | 51.1 | 82.4 | 91.3 | 8.23 |
| 3042051DR | 19.0 | 15.8 | 50.2 | 79.8 | 86.8 | 7.99 |
| 3042051RE | 19.0 | 16.4 | 51.3 | 80.3 | 87.7 | 7.99 |
| 3044647A | 19.0 | 15.9 | 47.1 | 74.2 | 83.6 | 7.41 |
| 3044647B | 19.0 | 15.6 | 48.1 | 75.2 | 88.1 | 7.89 |
| 3044647C | 19.0 | 16.0 | 47.2 | 72.8 | 83.8 | 7.70 |
| 3044647D | 19.0 | 15.5 | 47.9 | 74.1 | 83.7 | 7.48 |
| 3044647E | 19.0 | 16.9 | 49.7 | 75.0 | 82.9 | 7.48 |
| 3044669A | 18.0 | 14.6 | 43.5 | 75.1 | 85.6 | 7.57 |
| 3044669B | 18.0 | 17.6 | 52.3 | 83.1 | 89.8 | 8.19 |
| 3044669E | 18.0 | 16.1 | 48.7 | 77.8 | 82.1 | 7.71 |
| 3044646D | 17.0 | 17.1 | 50.2 | 78.4 | 84.7 | 7.67 |
| 3044646E | 17.0 | 16.3 | 49.1 | 77.2 | 84.4 | 7.61 |
| 3045325A | 13.0 | 16.1 | 47.2 | 71.0 | 75.2 | 6.86 |
| 3045325B | 13.0 | 14.1 | 41.5 | 66.4 | 73.9 | 6.55 |
| 3045325D | 13.0 | 16.3 | 45.9 | 70.3 | 74.1 | 6.75 |
| 3045325E | 13.0 | 15.2 | 43.9 | 66.3 | 71.0 | 6.39 |
| 3045326A | 22.0 | 15.2 | 48.4 | 74.3 | 85.6 | 7.40 |
| 3045326A | 22.0 | 15.8 | 49.0 | 79.7 | 89.2 | 7.99 |
| 3045326B | 22.0 | 17.1 | 51.4 | 77.8 | 85.4 | 7.59 |
| 3045326B | 22.0 | 14.5 | 47.2 | 79.1 | 88.9 | 8.08 |
| 3045326E | 22.0 | 15.2 | 47.3 | 79.0 | 88.0 | 7.99 |
| 3045327A | 17.0 | 17.7 | 51.6 | 80.0 | 86.6 | 7.80 |
| 3045327B | 17.0 | 17.8 | 55.0 | 84.9 | 89.8 | 8.38 |
| 3045327C | 17.0 | 18.6 | 52.3 | 80.T | 87.4 | 7.76 |
| 3045327D | 17.0 | 14.1 | 45.9 | 77.0 | 85.7 | 7.86 |
| 3045327E | 17.0 | 14.3 | 44.1 | 73.1 | 83.5 | 7.48 |
| 3045328A | 17.2 | 15.7 | 48.9 | 78.1 | 85.4 | 7.80 |
| 3045328B | 17.2 | 15.9 | 48.7 | 77.9 | 86.2 | 7.75 |
| 3045328C | 17.2 | 18.1 | 53.1 | 83.2 | 90.2 | 8.14 |
| 3045328D | 17.2 | 14.0 | 43.6 | 74.4 | 83.9 | 7.56 |
| 3045328E | 17.2 | 13.4 | 42.9 | 71.7 | 83.3 | 7.29 |
| 3045329A | 13.1 | 15.7 | 49.0 | 77.1 | 84.1 | 7.68 |
| 3045329B | 13.1 | 14.5 | 45.5 | 75.7 | 83.3 | 7.66 |
| 3045329C | 13.1 | 14.1 | 43.7 | 74.2 | 82.9 | 7.51 |
| 3045329D | 13.1 | 14.6 | 44.4 | 75.4 | 83.6 | 7.60 |
| 3045329E | 13.1 | 14.8 | 46.0 | 77.1 | 85.0 | 7.79 |
| 3045330C | 14.8 | 15.9 | 49.5 | 79.6 | 89.1 | 7.97 |
| 3045330D | 14.8 | 14.7 | 45.6 | 75.3 | 85.6 | 7.57 |
| 3045330E | 14.8 | 13.2 | 42.7 | 73.9 | 84.7 | 7.58 |

Table 2
Regression Analysis of Q vs. Fine
Regression Statistics

| | |
|---|---|
| Multiple R | 0.62403166 |
| R Square | 0.389415512 |
| Adjusted R | 0.373347499 |
| Square | |
| Standard Error | 4.706202075 |
| Observations | 40 |

ANOVA

| | | | | | Significance |
|---|---|---|---|---|---|
| | df | SS | MS | F | F |
| Re- | 1 | 536.7749241 | 536.774924 | 24.23545 | 1.68842E−05 |
| gression | | | | | |
| Residual | 38 | 841.6368428 | 22.14833797 | | |
| Total | 39 | 1378.411767 | | | |

| | Coefficients | Standard Error | t Stat | P-value |
|---|---|---|---|---|
| Intercept | 61.34159933 | 5.055611429 | 12.13336907 | 1.22E−14 |
| % Fines | 1.472116201 | 0.299031234 | 4.922951289 | 1.69E−05 |

Similar observations were found for the relationship between RR and % fines, as shown in Table 3. Again, the analysis demonstrates that the % fines has a statistically significant relationship on RR (t=4.4, F=19.4 and P<0.01).

TABLE 3
Regression analysis of RR vs. Fine
Regression Statistics

| | |
|---|---|
| Multiple R | 0.581735578 |
| R Square | 0.338416283 |
| Adjusted R | 0.321006185 |
| Square | |
| Standard Error | 0.460401873 |
| Observations | 40 |

ANOVA

| | | | | |
|---|---|---|---|---|
| | | | | Significance |
| df | SS | MS | F | F |

| | | | | | |
|---|---|---|---|---|---|
| Re- | 1 | 4.120256028 | 4.120256028 | 19.43793 | 8.24868E−05 |
| gression | | | | | |
| Residual | 38 | 8.054855621 | 0.211969885 | | |
| Total | 39 | 12.17511165 | | | |

| | Coefficients | Standard Error | t Stat | P-value |
|---|---|---|---|---|
| Intercept | 5.629807076 | 0.494584154 | 11.38291033 | 8.28E−14 |
| % Fine | 0.128975757 | 0.029253852 | 4.40884692 | 8.25E−05 |

Table 4 provides a side-by-side comparison of the dissolution characteristics of tablets produced using 39.2% fines and 21% fines in the drug layer.

TABLE 4
Dissolution Characteristics of Tablets Produced
Using 39.2% Fines Vs. 21% Fines in the Drug Layer

| | Wt. | | | | |
|---|---|---|---|---|---|
| Time | Gain | Batch with 39.2% fines | | Batch with 21% fines | |
| (mm) | (mg/tab) | Q | RR | Q | RR |
| 285 | 14.82 | 98.6 | 8.45 | 90.5 | 8.22 |
| 285 | 14.82 | 96.3 | 8.30 | 89.3 | 8.00 |
| 295 | 15.34 | 100.6 | 8.82 | 87.0 | 8.00 |
| 315 | 16.38 | 92.6 | 8.11 | 85.6 | 7.66 |
| 315 | 16.38 | 94.1 | 7.90 | 88.7 | 8.20 |
| 335 | 17.42 | 98.9 | 8.72 | 85.3 | 7.80 |
| 345 | 17.94 | 94.5 | 8.18 | 89.7 | 8.26 |
| 345 | 17.94 | 96.4 | 8.30 | 83.9 | 7.80 |
| Average | | 96.2 | 8.33 | 87.1 | 7.96 |

| | Q | RR | |
|---|---|---|---|
| Difference | 9.1 | 0.4 | Shasun > |
| | | | Calcium |

| Paired t-test for Q (% Dissolved at 16h) | |
|---|---|
| Critical t-two tail | t = 6.9 |
| | t = 2.4 |
| Paired t-test for RR (% Release Rate) | |
| Critical t-two tail | t = 2.4 |
| | t = 2.3 |

The results indicate that the increase in % fines in the drug layer corresponds to higher Q and higher RR values. In particular, the dissolution characteristics (Q and RR) of the tablets with 39.2% fines in the drug layer are significantly higher (t=6.9 and 2.4 for Q and RR, respectively) compared to batched with 21% fines in the drug layer.

In addition, Table 5 compares the % fines of the hydrophilic polymer and % fines of the drug layer, when the hydrophilic polymer, poly(ethylene oxide) (Polyox® N-80, Dow Chemical Co., Midland, Mich.), is used according to one embodiment of the present invention. The analytical results, shown in Table 6, demostrate that the % fines in the drug layer is significantly correlated with the particle size of the hydrophilic polymer (t=11.0, F=121.6, P<0.01).

TABLE 5
Polyox Fines and Drug Layer Fines

| | | % fines | % fines |
|---|---|---|---|
| Polyox | | Polyox | Drug |
| Lot# NCH | Dow Lot# | N80 | Layer |
| 10004564/10007114 | TB1855S5I3 | 28 | 35.2 |
| 10007114 | TB1855S5I3 | 28 | 31 |
| 10004564 | TB1855S5I3 | 28 | 37.3 |
| 10004564 | TB1855S5I3 | 28 | 35.9 |
| 10004564 | TB1855S5I3 | 28 | 39.2 |
| 10004564 | TB1855S5I3 | 28 | 36.5 |
| 10004564 | TB1855S5I3 | 28 | 36 |
| 10002416 | SJ1255S5I4 | 25 | 26.6 |
| 10002416 | SJ1255S5I4 | 25 | 24.3 |
| 10002415 | SJ1255S5I5 | 24 | 19.5 |
| 10002415 | SJ1255S5I5 | 24 | 16.7 |
| 10002415/10000668 | SJ1255S5I5 | 24 | 14.8 |
| 10002415 | SJ1255S5I5 | 24 | 13.1 |
| 10002415 | SJ1255S5I5 | 24 | 17.2 |
| 10002415 | SJ1255S5I5 | 24 | 17.1 |
| 10000668 | SJ1255S5I4 | 25 | 21.9 |
| 10000668 | SJ1255S5I4 | 25 | 13.4 |

TABLE 6
Regression Analysis of % Fines in Polyox vs. % Fines in Drug Layer
Regression Statistics

| | |
|---|---|
| Multiple R | 0.9435 |
| R Square | 0.8902 |
| Adjusted R | |
| Square | 0.8829 |
| Standard Error | 3.2846 |
| Observations | 17 |

ANOVA

| | | | | | Significance |
|---|---|---|---|---|---|
| | df | SS | MS | F | F |
| Re- | 1 | 1311.725 | 1311.725 | 121.5836 | 1.36149E−08 |
| gression | | | | | |
| Residual | 15 | 161.83 | 10.78867 | | |
| Total | 16 | 1473.555 | | | |

| | Coefficients | Standard Error | t Stat | P-value |
|---|---|---|---|---|
| Intercept | −99.9 | 11.41218 | −8.7538 | 2.79E−07 |
| Polyox | 4.85 | 0.43985 | 11.0265 | 1.36E−08 |

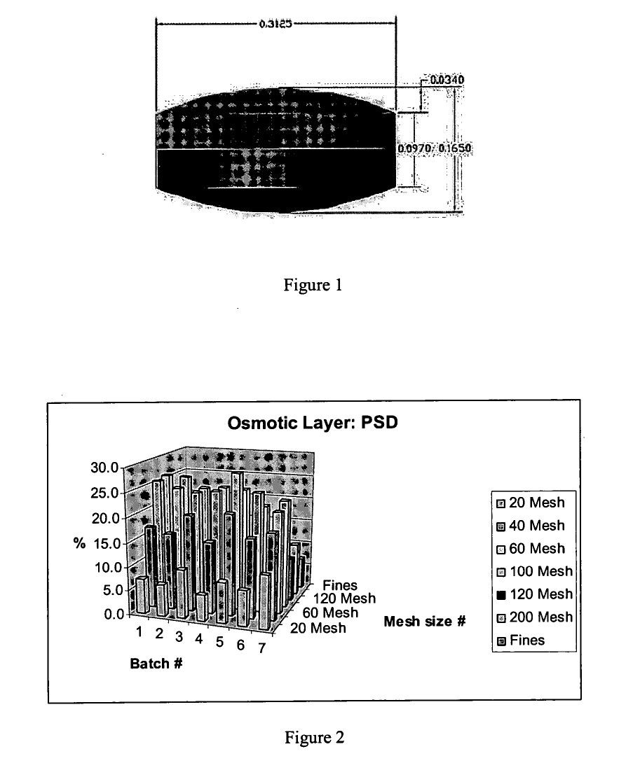

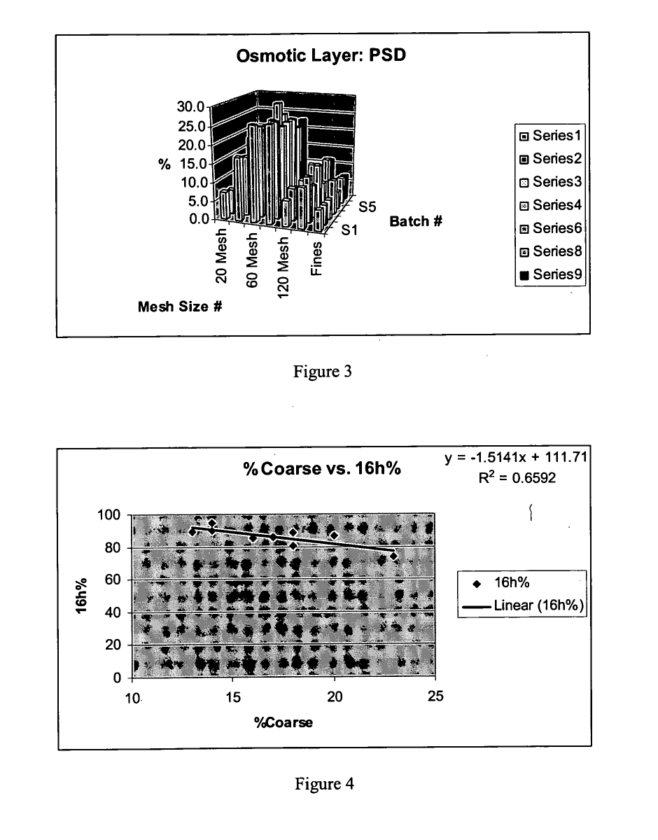

As shown in FIGS. 2 and 3, the particle size distributions (PSD) of all lots of osmotic granulations that were used to make the exemplary tablets discussed above do not show any significant difference among the batches, implying that the effect of osmotic layer particle size distribution is similar for all batches.

Accordingly, the above example demonstrates that % fines in the drug layer influences both the 16-hour cumulative drug release (Q) and the release rate (RR).

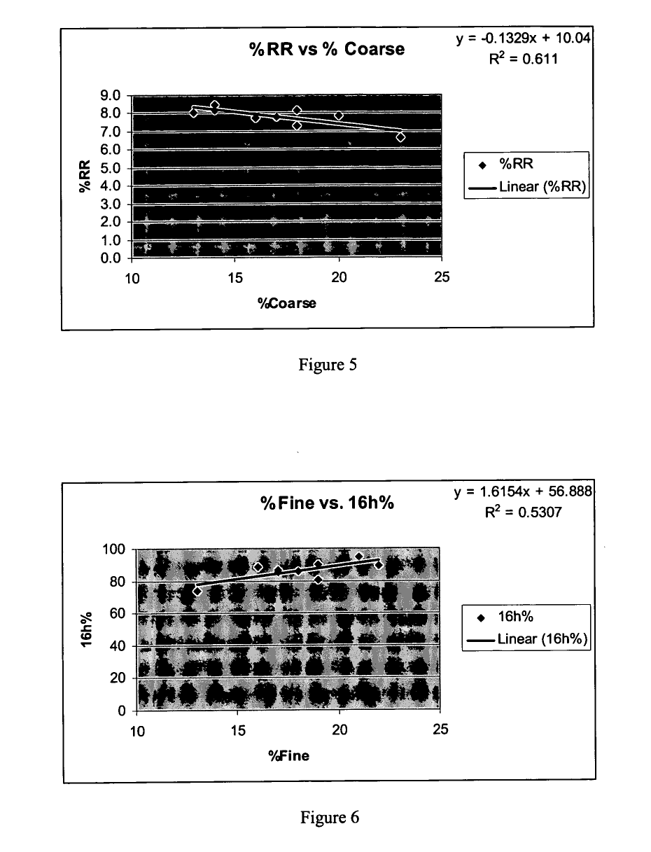

As shown in FIG. 4, effect of % coarse particles of the drug layer has been found to be significant (n=9, r²=0.67, t=−3.7) on Q. From the regression equation it can be stated that for every percent decrease in the amount of coarse particles there is a 1.5% increase in the dissolution at 16-hour time point (16 h %). In addition, as shown in FIG. 5, the effect of % coarse particles of the drug layer has been found to be significant (n=9, r²=0.61, t=−3.3) on % RR. From the regression equation it can be stated that for every percent decrease in the amount of coarse particles there is 0.13% increase in the dissolution rate (% RR).

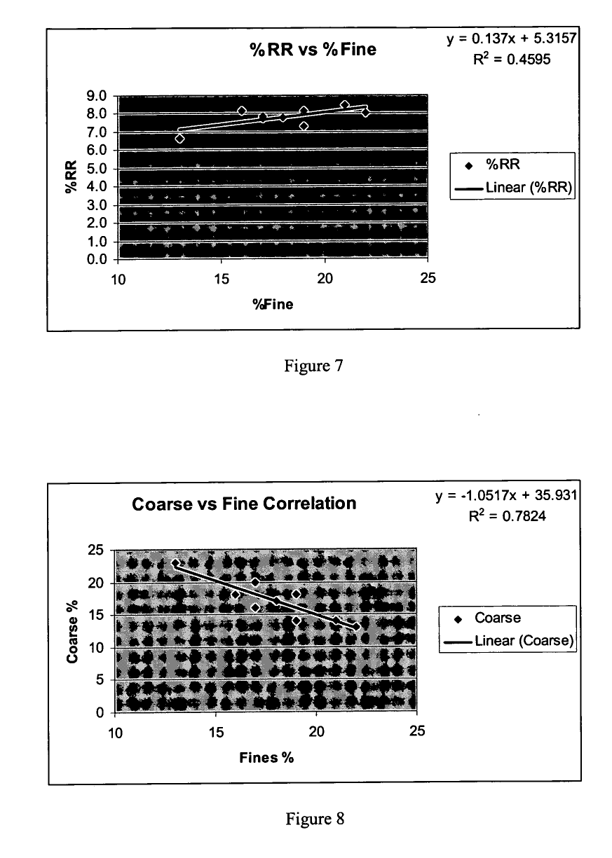

As shown in FIG. 6, and described in detail above, the % fines of the drug layer significantly affects Q (n=9, r²=0.53, t=2.8). From the regression equation it can be stated that for every percent increase in the amount of fine particles there is 1.6% increase in the dissolution at 16 h (16 h %). In addition, as shown in FIG. 7, and described in detail above, the % fine of the drug layer has been found to significantly affect % RR (n=9, r²=0.46, t=2.4). From the regression equation it can be stated that for every percent increase in the amount of fine particles there is 0.14% increase in the dissolution rate (% RR).

It has also been found that there is a direct and significant (t=−5.1) correlation between % fines and % coarse particles (n=9, r2=0.8), as shown in FIG. 8. Therefore, fines or coarse particles may be considered a single variable, rather than two separate variables.

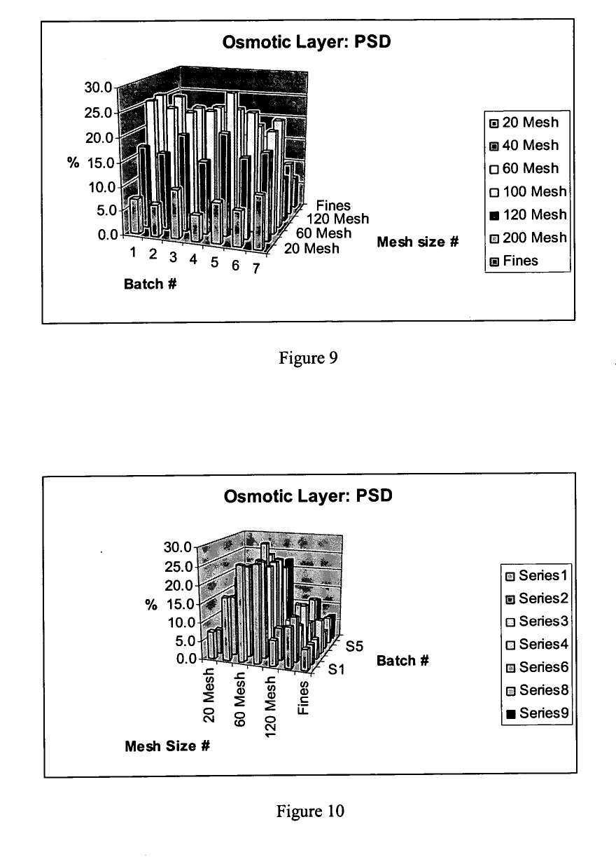

As shown in FIGS. 9 and 10, the particle size distributions (PSD) of 7 lots of osmotic granulations do not show any significant difference among the batches. Therefore, it is confirmed that the effect of due to differences in the osmotic layer is insignificant.

From the examples described above, it is demonstrated that the particle size of the drug layer, defined as fines, has a significant and unexpected impact on the 16-hour cumulative dissolution (Q) and the % Release Rate (RR).

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects.

1. An osmotic drug delivery system for delivering a drug to a subject comprising:
a drug layer comprising a drug and a carrier comprising a first hydrophilic polymer wherein the first hydrophilic polymer has a molecular weight of about 40,000 to about 750,000 and wherein the drug and the carrier are combined to form particles comprising from over 28% to about 100% fines;
an osmotic layer comprising a second hydrophilic polymer wherein the second hydrophilic polymer has a molecular weight of about 1,000,000 to about 15,000,000; and
an outer coating wherein the outer coating substantially surrounds the drug layer and the osmotic layer and wherein the outer coating is semi-permeable to allow fluid to enter the system and comprises at least one opening which allows the drug to be released from the drug layer.

2. (canceled)

3. The osmotic drug delivery system of claim 1 wherein the drug layer comprises over 28% to about 50% fines.

4. (canceled)

5. The osmotic drug delivery system of claim 1 wherein the drug layer comprises over 28% to about 40% fines.

6. The osmotic drug delivery system of claim 1 wherein the drug comprises a dihydropyridine.

7. The osmotic drug delivery system of claim 1 wherein the drug comprises isradipine.

8. The osmotic drug delivery system of claim 1 wherein the first hydrophilic polymer has a molecular weight of about 100,000 to about 300,000.

9. The osmotic drug delivery system of claim 1 wherein the second hydrophilic polymer has a molecular weight of about 5,000,000 to about 8,000,000.

10. The osmotic drug delivery system of claim 1 wherein at least one of the first and the second hydrophilic polymer comprises polyethylene oxide.

11. The osmotic drug delivery system of claim 1 wherein the drug delivery system releases at least 85% of the drug in 16 hours under the following dissolution conditions: USP dissolution test <711>, Apparatus 2, 50 rpm, 500 to 1000 ml of 0.2% N,N-dimethyl dodecylamine N-oxide (LDAO) or equivalent solvent, temperature 37° C.±0.5° C.

12. The osmotic drug delivery system of claim 1 wherein the drug delivery system releases at least 90% of the drug 16 hours under the following dissolution conditions: USP dissolution test <711>, Apparatus 2, 50 rpm, 500 to 1000 ml of 0.2% N,N-dimethyl dodecylamine N-oxide (LDAO) or equivalent solvent, temperature 37° C.±0.5° C.

13. The osmotic drug delivery system of claim 1 wherein the drug layer is comprised of at least 24% fines of hydrophilic polymer.

14. The osmotic drug delivery system of claim 1 wherein the drug layer is comprised of about 24% to about 50% fines of hydrophilic polymer.

15. The osmotic drug delivery system of claim 1 wherein the drug layer is comprised of at least 28% fines of hydrophilic polymer.

16. The osmotic drug delivery system of claim 1 wherein the drug layer is comprised of about 28% to about 50% fines of hydrophilic polymer.

17. A method of delivering a drug to a subject comprising administering to the subject an osmotic drug delivery system of claim 1.

18. A method of delivering isradipine to a subject comprising administering to the subject an osmotic drug delivery system comprising:
a drug layer comprising isradipine and a carrier comprising a first hydrophilic polymer, wherein the first hydrophilic polymer has a molecular weight of about 40,000 to about 750,000, and wherein the isradipine and the carrier are combined to form particles comprising from over 28% to about 100% fines;
an osmotic layer comprising a second hydrophilic polymer wherein the second hydrophilic polymer has a molecular weight of about 1,000,000 to about 15,000,000; and
an outer coating wherein the outer coating substantially surrounds the drug layer and the osmotic layer and wherein the outer coating is semi-permeable to allow fluid to enter the system and comprises at least one opening which allows the isradipine to be released from the drug layer.

19. The method of claim 18 wherein the drug delivery system releases at least 85% of the isradipine in 16 hours under the following dissolution conditions: USP dissolution test <711>, Apparatus 2, 50 rpm, 500 to 1000 ml of 0.2% N,N-dimethyl dodecylamine N-oxide (LDAO) or equivalent solvent, temperature 37° C.±0.5° C.

20. The method of claim 18 wherein the drug layer comprises over 28% to about 50% fines.

21. The osmotic drug delivery system of claim 1 wherein the drug layer comprises about 39% to about 100% fines.

22. The method of claim 18 wherein the drug layer comprises about 39% to about 100% fines.
